(19) United States
(12) Patent Application Publication
(10) Pub. No.: US 2023/0101064 A1
(43) Pub. Date: Mar. 30, 2023

(54) COMPOSITIONS SUITABLE FOR WHITENING ORAL CARE AND OTHER USES

(71) Applicant: RHODIA OPERATIONS, Lyon (FR)

(72) Inventors: Cédric Feral-Martin, Emerainville (FR); Caroline Fayolle, Lyon (FR); Anne-Laure Pinault, Fresnes (FR)

(73) Assignee: RHODIA OPERATIONS, Lyon (FR)

(21) Appl. No.: 17/952,078

(22) Filed: Sep. 23, 2022

Related U.S. Application Data
(63) Continuation-in-part of application No. PCT/EP2021/056566, filed on Mar. 15, 2021.

(30) Foreign Application Priority Data
Mar. 24, 2020 (EP) 20315044.6

(51) Int. Cl. A61K 8/81; A61K 8/25; A61K 8/86; A61K 8/27; A61K 8/43; A61K 8/22; A61Q 11/00

(52) U.S. Cl. CPC A61K 8/81; A61K 8/22; A61K 8/25; A61K 8/27; A61K 8/43; A61K 8/86; A61Q 11/00; A61K 2800/28; A61K 2800/524; B60C 1/0016

(57) ABSTRACT

A modified precipitated silica comprising precipitated silica and at least one polymer selected from the group consisting of poly(ethylene glycol) methyl ether methacrylate and poly(vinylphosphonic acid) is useful for the manufacture of peroxide-based whitening oral care compositions having improved stability over time. It is also useful for the manufacture of elastomeric compositions having a performing balance of properties including e.g. high rolling resistance and/or high wear resistance.

### TECHNICAL FIELD

This application is a continuation-in-part of international application No. PCT/EP2021/056566 filed on Mar. 15, 2021, which claims priority to European application 20315044.6 filed on Mar. 24, 2020, the whole content of these applications being incorporated herein by reference for all purposes.

The present invention relates to whitening oral care compositions comprising precipitated silica and having improved stability over time.

### BACKGROUND ART

Everyday activities, including eating or drinking, cause undesirable staining of surfaces of teeth. There is a variety of compositions described in the art for preventing or treating the discoloration of teeth. The materials most commonly used in teeth whitening today are peroxides. Peroxides are generally deemed safe from a physiological standpoint, and can be effective to whiten teeth.

Oral care compositions comprising peroxides or peroxide-releasing compounds are known. One drawback of the use of peroxides is their high reactivity, which leads to decomposition of the peroxide during storage hence to lower whitening activity of the composition. In addition to that, it has been observed that the presence of precipitated silica in the formulation may induce the decomposition of peroxide or peroxide-releasing compounds. Precipitated silica is used extensively in oral care formulations as abrasive and/or thickening agent.

For instance, precipitated silica abrasives provide controlled mechanical cleaning, plaque removal and polishing of teeth. Such silica abrasives are known to interact not only with peroxide or peroxide-releasing compound but also with other active ingredients of the compositions, such as certain antimicrobial agents, fluorides and zinc compounds. Such interactions have the consequence that these active ingredients are no longer available to elicit their beneficial effects.

It would thus be advantageous to obtain oral care compositions, comprising a peroxide or peroxide-releasing compound and precipitated silica, characterised by a reduced decomposition of the peroxide or peroxide-releasing compound, hence by a longer shelf life. It would also be advantageous to obtain oral care compositions containing precipitated silica and exhibiting at the same time reduced decomposition of peroxide or peroxide-releasing compound and higher compatibility with antimicrobial agents and/or zinc compounds.

Finally, it would also be advantageous that the precipitated silica that is useful in such oral care compositions be also useful for other important uses, especially as reinforcing filler in elastomeric compositions. Thus, in particular, it would be advantageous that the precipitated silica useful in such oral care compositions, when used in tire rubber compositions, provides a performing balance of properties, including e.g. at least one of a high dispersibility in a rubber matrix, a high rolling resistance, a high wear resistance, a high tensile strength and a high elongation at break.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a modified precipitated silica comprising precipitated silica and at least one polymer selected from the group consisting of poly(ethylene glycol) methyl ether methacrylate and poly(vinylphosphonic acid).

In another aspect, embodiments disclosed herein relate to an oral care composition comprising a peroxide-releasing compound and precipitated silica, characterized in that the precipitated silica is a modified precipitated silica comprising at least one polymer selected from the group consisting of poly(ethylene glycol) methyl ether methacrylate and poly(vinylphosphonic acid).

Other aspects and advantages of the claimed subject matter will be apparent from the following description and the appended claims.

### DETAILED DESCRIPTION

A first object of the invention is an oral care composition comprising a peroxide-releasing compound and precipitated silica, characterised in that the precipitated silica is a modified precipitated silica comprising at least one polymer selected from the group consisting of poly(vinylpyrrolidone), poly(ethylene glycol), poly(ethylene glycol) methyl ether methacrylate, and poly(vinylphosphonic acid).

The expression “peroxide-releasing compound” is used herein to refer to hydrogen peroxide, peroxides as well as any compound capable to release hydrogen peroxide under the conditions of use in an oral care application. Notable, non-limiting examples of peroxide-releasing compounds are include hydroperoxides, hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include urea peroxide, glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, and monoperoxyphthalate and mixtures thereof, as well as inorganic peroxy acid salts such as and perborate salts of alkali and alkaline earth metals such as lithium, potassium, sodium, magnesium, calcium and barium, and mixtures thereof. Preferred solid peroxides are sodium perborate, urea peroxide, and mixtures thereof.

The peroxide-releasing compound may be bound to a polymer such as polymers of poly(vinylpyrrolidone), polyacrylates, polymethacrylates.

The oral care composition typically contains from 1 to 50%, typically from 3 to 40%, preferably from 3 to 20% by weight of the peroxide-releasing compound.

The modified precipitated silica used in the inventive composition exhibits on its surface molecules of the at least one polymer as hereinafter defined. The at least one polymer is coated or adsorbed on the surface of the precipitated silica.

The expression “silica” is used herein to refer to silicon dioxide, SiO₂. The term “silica” is used throughout the text to refer to precipitated silica. The expression “precipitated silica” is used to refer to a synthetic amorphous silica obtained by a process wherein a silicate is reacted with an acid causing the precipitation of SiO₂.

The modified precipitated silica comprises precipitated silica and at least one polymer selected from the group consisting of poly(vinylpyrrolidone), poly(ethylene glycol), poly(ethylene glycol) methyl ether methacrylate and poly(vinylphosphonic acid).

The at least one polymer is preferably selected from the group consisting of poly(ethylene glycol) methyl ether methacrylate and poly(vinylphosphonic acid). The polymer is more preferably poly(vinylphosphonic acid).

The term “poly(vinylpyrrolidone)” is used herein to refer to polymers comprising recurring units deriving from N-vinyl-2-pyrrolidone (CAS number 9003-39-8). Suitable poly(vinylpyrrolidone) polymers typically have a molecular weight in the range from 2500 to 2500000 g/mol.

Suitable poly(ethylene glycol) methyl ether methacrylate polymers (CAS number 26915-72-0) typically have a molecular weight in the range from 300 to 10000 g/mol.

Suitable poly(ethylene glycol) polymers (CAS number 25322-68-3) typically have a molecular weight in the range from 200 to 20000 g/mol.

The term “poly(vinylphosphonic acid)” refers to polymers comprising recurring units deriving from vinyl phosphonic acid (CAS number 27754-99-0). Suitable poly(vinylphosphonic acid) polymers have a molecular weight in the range from 1550 to 45000 g/mol.

The modified silica typically comprises at least 1% by weight, preferably at least 2% by weight of the at least one polymer with respect to the weight of the modified silica. The modified silica may comprises up to 20% by weight, typically up to 15% by weight of the at least one polymer with respect to the weight of the modified silica. Suitable ranges are for instance from 1 to 12% by weight, even from 2 to 10% by weight of the at least one polymer with respect to the weight of the modified silica.

The presence of the at least one polymer on the surface of the modified silica may be determined, for instance, by extracting the compound with a suitable solvent and then performing routine analysis on the extract (e.g. by NMR).

The amount of the at least one polymer in the modified silica may also be measured by means of total carbon content. The amount of the at least one polymer in the modified silica, expressed as total carbon (C), is typically of at least 0.2 wt%, in particular of at least 0.4 wt%. Typically, the content, expressed as total carbon (C), does not exceed 20.0 wt%, in particular it does not exceed 15.0 wt%.

The modified precipitated silica may be either an abrasive silica or a thickening silica.

In a first embodiment of the oral care composition, the modified silica is an abrasive silica.

In such an embodiment, the modified silica has a CTAB surface area of at least 5 m²/g, typically at least 10 m²/g. The CTAB surface area does not generally exceed 90 m²/g. The CTAB surface area may be lower than 75 m²/g, preferably lower than 65 m²/g.

For applications as an abrasive in oral care formulations, advantageous ranges of CTAB surface area are from 10 to 70 m²/g, preferably from 15 to 65 m²/g.

The CTAB surface area is a measure of the external specific surface area as determined by measuring the quantity of N hexadecyl-N,N,N-trimethylammonium bromide adsorbed on the silica surface at a given pH. The CTAB surface area can be determined according to the standard NF ISO 5794-1, Appendix G (June 2010).

The BET surface area of the modified silica is not particularly limited but it is at least 5 m²/g, typically at least 10 m²/g. BET surface area may in certain instances be greater than 15 m²/g. BET surface area is generally at most 100 m²/g. The BET surface area may advantageously be from 8 to 85 m²/g, even from 10 to 70 m²/g, preferably from 15 to 65 m²/g.

The BET surface area is determined according to the Brunauer - Emmett -Teller method described in The Journal of the American Chemical Society, Vol. 60, page 309, February 1938, and corresponding to the standard NF ISO 5794-1, Appendix D (June 2010).

The modified precipitated silica is characterised by a good balance of abrasive properties, that is ability to remove the pellicle deposit of the teeth without damaging the enamel.

The modified silica is characterised by an abrasion depth value Hm, as determined using the PMMA abrasion test described hereafter, between 4.0 and 25.0 µm, preferably between 5.0 and 20.0 µm, more preferably between 5.0 and 15.0 µm.

When the modified silica is an abrasive silica, the oral care composition contains from 3 to 60%, typically from 5 to 50%, preferably from 5 to 30% by weight of the modified silica.

In a second embodiment of the oral care composition, the modified silica is a thickening silica.

In such an embodiment, the modified silica has a CTAB surface area of at least 90 m²/g, typically at least 100 m²/g. The CTAB surface area does not generally exceed 350 m²/g. The CTAB surface area may be lower than 300 m²/g, preferably lower than 250 m²/g.

For applications as an abrasive in oral care formulations, advantageous ranges of CTAB surface area are from 100 to 250 m²/g, preferably from 110 to 220 m²/g.

The BET surface area of the modified silica is not particularly limited but it is at least 100 m²/g, typically at least 110 m²/g. BET surface area may in certain instances be greater than 115 m²/g. BET surface area is generally at most 400 m²/g. The BET surface area may advantageously be from 100 to 350 m²/g, even from 120 to 300 m²/g, preferably from 120 to 275 m²/g.

When the modified silica is a thickening silica, the oral care composition contains from 1 to 50%, typically from 2 to 40%, preferably from 2 to 25% by weight of the modified silica.

The oral care composition may comprise a modified abrasive silica and/or a modified thickening silica.

The inventive composition of the invention may include other ingredients commonly used in oral care applications, in particular other water-insoluble inorganic abrasive agents, thickening agents, moisturizers, surfactants, and the like. Other abrasive agents which may be mentioned in particular are calcium carbonate, hydrated alumina, bentonite, aluminium silicate, zirconium silicate and sodium, potassium, calcium and magnesium metaphosphates and phosphates.

Among thickening agents mention may be made in particular of xanthan gum, guar gum, carrageenans, cellulose derivatives and alginates, in a quantity that can range up to 5% by weight of the composition.

Among the moisturizers mention may be made, for example, of glycerol, sorbitol, polyethylene glycols, polypropylene glycols and xylitol, in a quantity of the order of 2 to 85%, preferably of the order of 10 to 70% of the weight of composition, expressed on dry basis.

The inventive composition may additionally comprise surface-active agents, detergent agents, colorants, bactericides, fluorine derivatives, opacifiers, sweeteners, antitartar and antiplaque agents, sodium bicarbonate, antiseptics, enzymes, etc.

In a preferred embodiment of the invention, the composition further comprises antibacterial agent. Notable non-limiting examples of suitable antibacterial agents are chlorhexidine and chlorhexidine salts, such as bigluconate or diacetate, triclosan, cetylpyridinium chloride, benzalconium chloride and cetyltrimethylammonium bromide.

It has been observed that the use of a modified silica comprising at least one polymer as above detailed not only increases the stability of the inventive composition towards the loss of peroxide activity but it also increases the stability of antibacterial agents, and in particular antibacterial agents containing chlorhexidine.

Thus, in this embodiment of the invention, the oral care composition comprises a peroxide-releasing compound, an antibacterial agent containing chlorhexidine and a modified precipitated silica comprising at least one polymer selected from the group consisting of poly(vinylpyrrolidone), poly(ethylene glycol), poly(ethylene glycol) methyl ether methacrylate, and poly(vinylphosphonic acid).

The modified precipitated silica further exhibits a high compatibility with respect to cations which are customarily present in oral care compositions. Notable non limiting examples of said cations are for instance, calcium, strontium, barium, manganese, indium, nickel, zinc, titanium, zirconium, silver, palladium, ammonium or amino cations. These cations may be in the form of mineral salts, for example chloride, fluoride, nitrate, phosphate, sulfate or in the form of organic salts such as acetates, citrates.

In a further embodiment of the invention, the oral care composition comprises a peroxide-releasing compound, an antimicrobial agent containing chlorhexidine, a Zn containing salt and a modified precipitated silica whose surface is coated with at least one polymer selected from the group consisting of polyvinylpyrrolidone, poly(ethylene glycol) methyl ether methacrylate, and poly(vinylphosphonic acid).

Advantageously, the inventive composition has a compatibility with zinc, as determined using the Zn compatibility method described hereafter, of at least 50%.

In preferred embodiments of this invention, the oral composition is a dentifrice. Such dentifrices may include toothpaste (dental cream), tooth powders, or gel, or any other form known to one skilled in the art.

The inventive oral care composition may be used for the cleaning of teeth.

A second object of the invention is a modified precipitated silica which comprises precipitated silica and at least one polymer selected from the group consisting of poly(ethylene glycol) methyl ether methacrylate and poly(vinylphosphonic acid).

The precipitated silica may be any precipitated silica which is suitable for use as an abrasive silica in oral care application.

Notable non-limiting examples of suitable commercially available precipitated silicas are for instance : Tixosil® 73, Tixosil® 63, Tixosil® SoftClean, Tixosil® 43, Tixosil® 331 all available from Solvay SA.

The modified silica typically comprises at least 1% by weight, preferably at least 2% by weight of the at least one polymer with respect to the weight of the modified silica. The modified silica may comprises up to 20% by weight, typically up to 15% by weight of the at least one polymer with respect to the weight of the modified silica. Suitable ranges are for instance from 1 to 12% by weight, even from 2 to 10% by weight of the at least one polymer with respect to the weight of the modified silica.

The modified silica of the present invention may be prepared by any process suitable for coating or adsorbing a polymer on the surface of precipitated silica.

In a first embodiment, the process for the preparation of the modified silica comprises the steps of:
providing a precipitated silica; and
adsorbing at least one polymer as defined above on said precipitated silica.

The silica may be in any form, such as a powder, granules, or substantially spherical beads. Adsorption may be carried out according to any means known in the art.

Adsorption may be obtained by impregnating granules or beads of the silica with the at least one polymer in the molten state. Alternatively, the at least one polymer may be dispersed or dissolved in a suitable liquid carrier.

The step of impregnating the precipitated silica with the at least one polymer may be carried out using any suitable equipment. For instance, molten polymer, its dispersion or its solution may be sprayed onto the silica maintained under suitable agitation. A mixer or an internal blender of the Brabender type may be used for the impregnation.

After the molten polymer, its dispersion or its solution, has been contacted with the silica, drying may be optionally carried out. Drying may be particularly advantageous when the at least one polymer is in the form of a dispersion or solution in a liquid carrier, either aqueous or organic. In this latter case the solvent is typically removed by evaporation.

In a further embodiment, the process for the preparation of the modified silica comprises the steps of:
reacting at least one silicate with at least one acid, to provide a silica suspension;
submitting said silica suspension to filtration to provide a filter cake;
submitting said filter cake to a liquefaction step to obtain a suspension of precipitated silica;
adding at least one polymer as above defined to the filter cake before, during or after the liquefaction step
optionally, drying the suspension of precipitated silica obtained after the liquefaction step to obtain modified precipitated silica.

Several methods can be employed for the precipitation of silica: notably, the addition of an acid to a solution of the silicate, or simultaneous addition, partial or total, of an acid and of the silicate to water or to a silicate solution already present in the vessel.

At the end of the precipitation reaction, a suspension of precipitated silica is obtained, which is subsequently separated (liquid/solid separation). The process in all of its embodiments, thus typically comprises a further step of filtering the suspension of precipitated silica and drying the precipitated silica.

The separation usually comprises a filtration, followed by washing, if necessary. The filtration is performed according to any suitable method, for example by means of a belt filter, a rotary filter, for example a vacuum filter, or, preferably a filter press.

The filter cake is then subjected to a liquefaction operation. The term “liquefaction” is intended herein to indicate a process wherein a solid, namely the filter cake, is converted into a fluid-like mass. After the liquefaction step the filter cake is in a flowable, fluid-like form and the precipitated silica is in suspension.

The liquefaction step may comprise a mechanical treatment which results in a reduction of the granulometry of the silica in suspension. Said mechanical treatment may be carried out by passing the filter cake through a high shear mixer, a colloidal-type mill or a ball mill. Optionally, the liquefaction step may be carried out by subjecting the filter cake to a chemical action, for instance by addition of water or an acid. The mechanical and chemical treatments may be both carried out.

The at least one polymer can be added to the filter cake before, during or after the liquefaction step. Typically, the at least one polymer is added to the filter cake during or after the liquefaction step, either as a solid or as a liquid, dispersion or solution.

Notable, non-limiting examples of suitable processes for the preparation of precipitated silica are disclosed for instance in EP396450A, EP520862A, EP647591A, EP670813A, EP670814A, EP901986A, EP762992A, EP762993A, EP917519A, EP983966A, EP1355856A, WO03/016215, WO2009/112458, WO2011/117400.

A further object of the present invention is a method for reducing the decomposition of peroxide-releasing compounds in an oral care composition comprising a peroxide-releasing compound and precipitated silica, said method characterized in that the precipitated silica is a modified precipitated silica comprising at least one polymer selected from the group consisting of poly(vinylpyrrolidone), poly(ethylene glycol), poly(ethylene glycol) methyl ether methacrylate, and poly(vinylphosphonic acid).

The use of the modified precipitated silica as above detailed allows obtaining peroxide-containing compositions which retain their peroxide activity over a long period of time.

Additionally the use of the modified precipitated silica allows at the same time to maintain high over time the availability of both chlorhexidine based antibacterial agents as well as zinc based compounds.

A last object of the present is an elastomeric composition comprising at least one elastomer and the modified precipitated silica. This last object concerns also a use of the modified precipitated silica as a reinforcing filler of an elastomeric composition, otherwise said it concerns also a method for increasing the strength of an elastomeric composition comprising at least one elastomer, said method comprising mixing the modified precipitated silica with the elastomer. As will be detailed later on, it concerns also an article comprising an elastomeric composition comprising at least one elastomer and the modified precipitated silica.

The elastomer has advantageously at least one glass transition temperature between -150° C. and +300° C., preferably between -150° C. and +20° C.

Certain suitable elastomers comprise repeat units derived from aliphatic or aromatic monomers comprising at least one unsaturation, such as ethylene, propylene, butadiene, isoprene, styrene, acrylonitrile, isobutylene or vinyl acetate, polybutyl acrylate and mixtures thereof. Other suitable elastomers comprise repeat units derived from chloro- or bromobutyl monomers (like bromo-butylene). Mention may also be made of functionalized elastomers, that is elastomers functionalized by chemical groups positioned along the macromolecular chain and/or at one or more of its ends (for example by functional groups capable of reacting with the surface of the silica), and halogenated polymers.

Certain suitable elastomers comprise repeat units derived from dienes, that is to say aliphatic or aromatic monomers comprising two and only two unsaturations. Exemplary diene elastomers mention include polybutadienes (BRs), polyisoprenes (IRs), butadiene copolymers, isoprene copolymers and mixtures thereof, in particular styrene/butadiene copolymers [SBRs, in particular ESBRs (emulsion) or SSBRs (solution)], isoprene/butadiene copolymers (BIRs), isoprene/styrene copolymers (SIRs), isoprene/butadiene/styrene copolymers (SBIRs), ethylene/propylene/diene terpolymers (EPDMs), and also the associated functionalized polymers, which can exhibit, for example, pendant polar or reactive groups or polar groups at the chain end, capable of interacting or reacting with the silica.

Among suitable elastomers, mention may also be made of natural rubber (NR) and epoxidized natural rubber (ENR).

The elastomeric composition may further comprise at least one silicapolymer coupling agent. Non-limiting examples of suitable coupling agents are for instance “symmetrical” or “unsymmetrical” silane polysulfides; mention may more particularly be made of bis((C1-C4)alkoxyl(C1-C4)alkylsilyl(C1-C4)alkyl) polysulfides (in particular disulfides, trisulfides or tetrasulfides), such as, for example, bis(3-(trimethoxysilyl)propyl) polysulfides or bis(3-(triethoxysilyl)propyl) polysulfides, such as triethoxysilylpropyl tetrasulfide. Mention may also be made of monoethoxydimethylsilylpropyl tetrasulfide. Mention may also be made of silanes comprising masked or free thiol functional groups (like NXT™ or NXT™ Z45 silanes), of mercaptopropyltriethoxysilane, and of a mixture mercaptopropyltriethoxysilane+octyltriethoxysilane (like SI 363® from Evonik). The coupling agent may be combined with a coupling activator, that is to say a compound which, when mixed with this coupling agent, increases the effectiveness of the latter.

The modified precipitated silica can advantageously act as a reinforcing filler of the elastomeric composition. In some preferred embodiments, the modified precipitated silica is the sole reinforcing filler of the elastomeric composition. In other embodiments, the elastomeric composition may further comprise at least one silica other than the modified precipitated silica and/or at least one organic or inorganic reinforcing filler other than a silica. Non limitative examples of silicas other than the modified precipitated silica are Zeosil® Premium SW, Zeosil® Premium 200MP, Zeosil® 1165MP, Zeosil® 1115MP and Zeosil®1085 GR, commercially available from Solvay SA. Non limitative examples of inorganic reinforcing inorganic fillers other than silica are nanoclays and alumina. Non limitative examples of organic reinforcing fillers are carbon black, carbon nanotubes, graphene, starch and cellulose. In such other embodiments, the modified precipitated silica preferably constitutes at least 30% by weight, preferably at least 60%, indeed even at least 80% by weight, of the total amount of the reinforcing filler.

The elastomeric composition may also comprise at least one other additive, such as an accelerator (like N-cyclohexyl-2-benzothiazole sulfonamide), a vulcanizing or crosslinking agent (like sulphur or a peroxide), a processing oil, an activator (like stearic acid or zin oxide), a processing aid (like a fatty acid or a zinc soap, a wax (like a PE wax), an antioxidant, a UV protector or an antiozonant.

The elastomeric composition may be a vulcanized composition (wherein the vulcanization has been made e.g. with the involvement of sulfur) or a crosslinked composition (wherein the crosslinking has been made e.g. with the involvement of a peroxide or another crosslinking system such as a diamine or a phenolic resin).

The amount of the modified precipitated silica in the elastomeric composition can vary within a broad range, depending notably on the specifically intended use of said elastomeric composition. It ranges generally from 1 wt% to 250 wt%, in particular from 5 wt% to 200 wt%, especially from 10 wt% to 170 wt%, for example from 20 wt% to 140 wt% or even from 25 wt% to 130 wt%, or alternatively from 10 wt% to 40 wt%, based on the weight of the at least one elastomer (the present wt% are sometimes referred to as “phr” or “Per Hundred Rubber” as they are based on 100 parts by weight of the at least one elastomer).

The elastomeric composition is useful for the manufacture of a number of semi-finished or finished articles. Non-limiting examples of articles comprising, consisting essentially of or consisting of the elastomeric composition are a footwear sole, a shoe, a floor covering layer, a floor covering, a tire part and a tire.

The elastomeric composition is especially useful for the manufacture of a tire, in particular of a tire part, more particularly of a tire tread. Tires, tire parts and tire treads comprising the elastomeric composition, tire parts and tire treads consisting essentially of the elastomeric composition, and tire parts and tire treads consisting of the elastomeric composition, exhibit advantageously a performing balance of properties, including e.g. at least one of a high dispersibility in a rubber matrix, a high rolling resistance, a high wear resistance, a high tensile strength and a high elongation at break.

Should the disclosure of any patents, patent applications, and publications which are incorporated herein by reference conflict with the description of the present application to the extent that it may render a term unclear, the present description shall take precedence.

The invention will be now described in more detail with reference to the following examples whose purpose is merely illustrative and not limitative of the scope of the invention.

### Analytical Methods

The physicochemical properties of the precipitated silica of the invention were determined using the methods described hereafter.

### Determination of Peroxide Stability

The compatibility of silica with a hydrogen peroxide solution was determined by the method described below.

A solution of URECO HC L17 (molecular weight = 277.18 g/mol; 17 wt% in hydrogen peroxide), an organic peroxide-releasing compound, at 2 wt% was prepared by dispersion of the solution into water. The hydrogen peroxide solution used was (solution A).

Starting from solution A, a suspension containing 10 wt% of silica, 2 wt% of hydrogen peroxide was prepared. The suspension thus obtained was stirred for 7 days at 25° C. (suspension B).

Between 3 and 4 g of the suspension maintained under agitation were placed in a bottle which can be closed. After sampling, 2 g of KI and 5 mL of acetic acid (99 wt%) were added to the suspension. The bottle was closed and protected from light and the reaction between hydrogen peroxide and the iodide allowed to proceed for 25 minutes.

A iodometry dosing was then performed on suspension B. The unreacted iodine was dosed using a sodium thiosulfate solution at 0.1 mol/L with a Pt titrode.

The hydrogen peroxide compatibility was calculated as the ratio of peroxide hydrogen available in suspension B with respect to the initial value according to the following formula:

$$H 202 c o m p a t i b i l i t y \left(\%\right) = \frac{H 202 i n s u s p e n s i o n \text{} B}{H 202 i n s o l u t i o n A} \times 100$$

### Determination of Chlorhexidine Compatibility

The compatibility of silica with chlorhexidine was determined using a modification of the method disclosed in EP315503B1. The method was performed as follows:
(1) a solution of chlorhexidine digluconate at 1 wt% was prepared by dispersion of the solution into water;
(2) 4 g of silica were dispersed in 16 g of the solution obtained in (1). The suspension thus obtained was stirred for 24 hours at 40° C.
(3) The suspension was filtered through a 0.45 µm PVDF filter.
(4) 0.5 mL of the filtered solution was diluted in 100 mL.

The concentration of chlorhexidine was measured in the solution prepared in (1) and in the supernatant obtained in (3) using the value of the absorbance of the two solutions at 254 nm measured using a UV-vis spectrophotometer (Uvikon 810/820).

Chlorhexidine compatibility was calculated as the ratio of chlorhexidine ions available in the solution obtained at the end of step (3) with respect to the theoretical value according to the following formula:

$$C h l o r h e x i d i n e c o m p a t i b i l i t y \left(\%\right) = \frac{C h l o r h e x i d i n e i n s u p e r n a t a n t \left(\left(3\right)\right)}{C h l o r h e x i d i n e i n s o l u t i o n \left(\left(1\right)\right)} \times 100$$

### Determination of Zn(II) Ion Compatibility

The compatibility of precipitated silica with Zn(II) ions was determined using a modification of the method disclosed in EP2349488B1.

The method was performed as follows:
(1) a solution of ZnSO₄.7 H₂O at 0.06%wt was prepared by dissolution of the solid into water
(2) 4 g of silica were dispersed in 100 mL of the solution obtained in (1).
The suspension thus obtained was stirred for 24 hours at 40° C.
(3) The suspension was filtered through a 0.45 µm PVDF filter.
(4) The concentration of Zn(II) was measured in the solution prepared in (1) and in the supernatant obtained in (3) using inductively coupled plasma (ICP-OES) using a PlasmaQuant PQ9000 Elite instrument.

The Zn(II) compatibility was calculated as the ratio of Zn(II) ions available in the solution obtained at the end of step (3) with respect to the theoretical value according to the following formula:

$$\text{Zn} \left(\text{II}\right) \text{compatiblity} \left(\%\right) = \frac{\left(\text{Zn} \left(\text{II}\right) i n s u p e r n a t a n t \left(3\right)\right)}{\left(\text{Zn} \left(\text{II}\right) i n s o l u t i o n \left(1\right)\right)} \times 100$$

### Determination of Abrasion Depth Hm

Abrasivity of silica was determined according to an internal method using poly(methyl methacrylate) (PMMA) plates as a substrate. Method is correlated to state of the art abrasivity test Relative Dentine Abrasion (RDA) (F. Peditto et al. “Which Alternatives to ISO11609 RDA? PMMA Abrasion Test with Silicas”, IADR GA Seattle, 2013).

Cast PMMA plates (Altuglas CN, Atoglas, Shore D hardness 60-70) 89 x 20 x 7.5 mm were used as substrate. On each plate a 3 mm wide zone for brushing (Testing area) was defined using adhesive tape and then sumitted to brushing for 10000 cycles using toothbrushes Brosserie Française, held at 15° angle and under a 240 g load, in the presence of slurries of abrasive silica prepared according to ISO1 1609:2010 protocol. The abrasion depth (Hm, expressed in µm) at the end of the brushing cycles was measured across a 20 x 10 mm area including the Testing area by optical profilometry (Altimet Altisurf 500) on rinsed plates. The area around the Testing area was used to define the baseline for the optical profilometry determination.

### Determination of Optical Properties

The optical properties of the modified silica were measured on a silica suspension in a water-sorbitol solution using a UV-Vis spectrophotometer. Each suspension was prepared by mixing 1 g of silica in 19 g of water-sorbitol solution. The of the silica was measured in sorbitol. The reported value of the refractive index corresponds to the refractive index at the wavelength where the suspension is the most transparent (maximum of transmittance). The transmittance value corresponds to the transmission at 589 nm.

### Determination of Carbon Content

The content of carbon was measured using a carbon/sulfur analyzer, such as the Horiba EMIA 320 V2. The principle of the carbon/sulfur analyzer is based on the combustion of a solid sample in a stream of oxygen in an induction furnace (adjusted to approximately 170 mA) and in the presence of combustion accelerators (approximately 2 grams of tungsten (in particular Lecocel 763-266) and approximately 1 gram of iron). The carbon present in the sample to be analyzed (weight of approximately 0.2 gram) combines with the oxygen to form CO₂, CO. These gases are subsequently analyzed by an infrared detector. The moisture from the sample and the water produced during these oxidation reactions is removed by passing over a cartridge comprising a dehydrating agent (magnesium perchlorate) in order not to interfere with the infrared measurement. The result is expressed as weight of element carbon per weight of SiO₂.

### Examples

Materials:
CS1: Precipitated silica Tixosil® 73 commercially available from Solvay SA
CS2: Precipitated silica Tixosil®63 commercially available from Solvay SA
PVP : Poly(vinylpyrrolidone); average molecular weight 1,300,000 g/mol; Sigma-Aldrich.
PEG-MEM : poly(ethylene glycol) methyl ether methacrylate, average weight 2000 g/mol, Sigma-Aldrich
PVPA : poly(vinylposphonic acid) average molecular weight 24000 g/mol ; Sigma-Aldrich

### Example 1

CS1 was used as a starting material for the preparation of a modified silica. An ethanol solution (at 3 wt%) of PEG-MEM was used. 300 g of CS1 were placed in a blade mixer (Gebrüder lodige Maschinenbau GmbH D-33102, volume 5 L) and the solution of PEG-MEM was injected into the mixer operating at 150 rpm through a nozzle at a pressure of 2 bars and at 50° C. The injection operation was carried out to achieve a PEG-MEM/SiO₂ weight ratio of 3%.The addition time was 7 minutes. Once the impregnation operation was finished, the equipment was stopped. The modified silica was recovered and placed in an oven at 35° C. during 12 h. The properties of inventive silica S1 and of originating silica CS1 are reported in Table 1.

### Example 2

Modified silica S2 was prepared starting from CS1 and an ethanol solution of PVP (3 t%) following the same procedure of Example 1. The PVP/SiO₂ ratio at the end of the impregnation phase was 3 wt%. The properties of modified silica S2 are reported in Table 1.

### Example 3

Modified silica S3 was prepared starting from CS1 and an ethanol solution of PVP (3 wt%) following the same procedure of Example 1. The PVA/SiO₂ ratio at the end of the impregnation phase was 3 wt%. The properties of modified silica S3 are reported in Table 1.

### Example 4

A silica suspension of CS1 in water was prepared. The silica suspension was filtered and washed on a drum filter to obtain a filter cake. A solution in water of PEG-MEM (50 wt%) was prepared. 805.6 g of filter cake of CS1 was subjected to a liquefaction operation in a continuous vigorously stirred reactor with simultaneous addition to the cake of 17 grams of the PEG-MEM solution to achieve a PEG-MEM/SiO₂ ratio of 3.0 wt%). 164.5 g of water were added to obtain a moisture equal to 30%. The disintegrated cake was subsequently dried using a nozzle atomizer by spraying under inert atmosphere. The properties of modified silica S4 are reported in Table 1.

### Example 5

The procedure described in Example 4 was followed to prepare a modified silica S5 starting from CS1 and a solution in water of PVA to achieve a PVA/SiO₂ ratio of 3.0 wt%). The properties of modified silica S5 are reported in Table 1.

### Example 6

Following the procedure of Example 1 the following modified silica S6 to S8 were prepared starting from CS2:
S6 : PEG-MEM/SiO₂ ratio of 3 wt%
S7 : PVA/SiO₂ ratio 3 wt%
S8 : PVA/SiO₂ ratio 6 wt%

The properties of modified silica S6, S7, S8 are reported in Table 1.

TABLE 1

| | CTAB (m²/g) | Moisture (%) | C content (%) | Compatibility peroxide - 7 days at 25° C. (%) | PMMA abrasivity Hm [µm] | Refractive index | %T max |
|---|---|---|---|---|---|---|---|
| CS1 | 50 | 6.82 | - | 47 | 8.7 | 1.440 | 82 |
| CS2 | 35 | 7.00 | - | 55 | - | 1.440 | 82 |
| S1 | 48 | 6.89 | 1.4 | 89 | 9.8 | - | - |
| S2 | 50 | 7.4 | 0.9 | 85 | - | - | - |
| S3 | 47 | 6.87 | 2.8 | 100 | 13.1 | 1.440 | 83 |
| S4 | 46 | 5 | 1.2 | 75 | - | - | - |
| S5 | 49 | 13.15 | 0.6 | 92 | - | - | - |
| S6 | 35 | 6.98 | 1.5 | 90 | - | 1.440 | 76 |
| S7 | 32 | 7.05 | 0.8 | 100 | - | 1.440 | 82 |
| S8 | 33 | 6.99 | 1.6 | 100 | - | 1.442 | 82 |

The hydrogen peroxide compatibility data in Table 1 show that modified silica S1 to S8 reduce the activity of the peroxide over time much less than the non-modified silica CS1 or CS2.

The modified silica maintains the same or even a higher level of abrasivity as the reference (S3 vs CS1).

Under the testing conditions employed, the optical properties of the modified silica remain unchanged with respect to the reference. This allows to replace precipitated silica with the modified silica without affecting the appearance of the oral care composition

### Example 9

In a 170 L stainless steel reactor were introduced: 17.8 L of water and 7 kg of a sodium silicate solution (SiO₂/Na₂O ratio = 3.44; SiO₂ concentration = 12 wt%). The same sodium silicate solution was used in all the steps of the process. The obtained solution was stirred and heated to reach 90° C. Once the set temperature was reached sulfuric acid (7.7 wt% solution) was added at a flowrate of 491 g/min until the reaction medium reached the pH value of 9.0. The same sulfuric acid solution was used in all the steps of the process. Simultaneously, over a period of 60 min, were introduced: sodium silicate, at a flowrate of 1429 g/min, and sulfuric acid. The flowrate of the sulfuric acid was regulated so that the pH of the reaction medium was maintained at a value of 9.0. At the end of the simultaneous addition, the pH of the reaction medium was brought to a value of 7.0 sulfuric acid. Simultaneously, the reaction medium was heated to 95° C. The rest of the process was carried out at this temperature. A first ageing step was carried out at pH 7.0 over a period of 75 min. After 75 min, the pH of the reaction medium was brought to a value of 4.0 with sulfuric acid at a flowrate of 680 g/min. At pH 4.0, a second ageing step was carried out over a period of 10 min to obtain a suspension of precipitated silica. The suspension of precipitated silica was filtered and washed on a filter plate. The moisture of the cake was more than 30 wt%. The filter cake obtained was disintegrated mechanically and water was added to obtain a SiO₂ suspension having 30 wt% of silica content. The product was dried by spray drying. The product obtained, in powder form, had a moisture content of less than 7 wt%.

The physicochemical properties of reference silica CS3 are reported in Table 2.

### Example 10

Following the procedure of Example 1 the following modified silica S9 to S10 were prepared starting from CS3:
S9 : PVA/SiO₂ ratio 3 wt%
S10 : PVA/SiO₂ ratio 6 wt%

TABLE 2

| | CTAB (m²/g) | C content (%) | Chlorhexidine Comp. (%) | Zn Comp. (%) | PMMA abrasion Hm [µm] | Refractive index | %T max |
|---|---|---|---|---|---|---|---|
| CS3 | 60 | - | 70 | 70 | 10.9 | 1.446 | 100 |
| S9 | 40 | 0.95 | 91 | 97 | 15.4 | 1.446 | 100 |
| S10 | 38 | 1.3 | 89 | 97 | | | |

The modified silica also exhibit higher compatibility with chlorhexidine and with Zn with respect to a unmodified silica.

Under the testing conditions employed, the optical properties of the modified silica remain unchanged with respect to the reference. This allows to replace precipitated silica with the modified silica without affecting the appearance of the oral care composition.

1. A modified precipitated silica comprising precipitated silica and at least one polymer selected from the group consisting of poly(ethylene glycol) methyl ether methacrylate and poly(vinylphosphonic acid).

2. The modified silica of claim 1 which comprises 1% to 20% by weight of the at least one polymer with respect to the weight of the modified silica.

3. The modified silica of claim 1 wherein the at least one polymer is poly(vinylphosphonic acid).

4. The modified silica of claim 3 which comprises 1% to 10% by weight of poly(vinylphosphonic acid) with respect to the weight of the modified silica.

5. A process for the manufacture of the modified precipitated silica of claim 1,
said process comprising the steps of: providing a precipitated silica; and
adsorbing at least one polymer selected from the group consisting of poly(ethylene glycol) methyl ether methacrylate and poly(vinylphosphonic acid) on said precipitated silica.

6. The process according to claim 5 which comprises the step of preparing a precipitated silica by a precipitation reaction between a silicate and an acidifying agent, to obtain a silica suspension, recovering the silica from the suspension and drying the silica.

7. A process for the preparation of the modified precipitated silica of claim 1 which comprises the steps of:
reacting at least one silicate with at least one acid, to provide a silica suspension;
submitting said silica suspension to filtration to provide a filter cake;
submitting said filter cake to a liquefaction step to obtain a suspension of precipitated silica;
adding at least one polymer selected from the group consisting of poly(ethylene glycol) methyl ether methacrylate and poly(vinylphosphonic acid) to the filter cake before, during or after the liquefaction step; and
optionally, drying the suspension of precipitated silica obtained after the liquefaction step to obtain modified precipitated silica.

8. A method for reducing the decomposition of peroxide-releasing compounds in an oral care composition comprising a peroxide-releasing compound and a precipitated silica, wherein the precipitated silica is the modified precipitated silica according to claim 1.

9. An oral care composition comprising a peroxide-releasing compound and precipitated silica, characterized in that the precipitated silica is a modified precipitated silica comprising at least one polymer selected from the group consisting of poly(ethylene glycol) methyl ether methacrylate and poly(vinylphosphonic acid).

10. The oral care composition according to claim 9 further comprising an antibacterial agent containing chlorhexidine and/or a Zn containing salt.

11. The oral care composition according to claim 9 wherein the at least one polymer is poly(vinylphosphonic acid).

12. The oral care composition of claim 9 which contains from 3 to 60% by weight of the modified precipitated silica and wherein the modified precipitated silica is an abrasive silica.

13. The oral care composition of claim 9 which contains from 1 to 50% by weight of the modified precipitated silica and wherein the modified precipitated silica is a thickening silica.

14. A method for preventing or treating the discoloration of a tooth which comprises contacting the oral care composition of claim 9 with the tooth.

15. An elastomeric composition comprising at least one elastomer and the modified precipitated silica of claim 1.

16. The elastomeric composition of claim 15 wherein the modified precipitated silica comprises precipitated silica and poly(vinylphosphonic acid).

17. The elastomeric composition of claim 15 wherein the amount of the modified precipitated silica ranges from 25 wt% to 130 wt%, based on the weight of the at least one elastomer.

18. An article comprising the elastomeric composition of claim 15.

19. The article of claim 18 which is selected from the group consisting of footwear soles, shoes, floor covering layers, floor coverings, tire parts and tires.

20. The article of claim 19 which is a tire tread.
